(19) United States
(12) Patent Application Publication
(10) Pub. No.: US 2013/0281852 A1
(43) Pub. Date: Oct. 24, 2013

(54) METHOD FOR CORRECTING BLOOD PRESSURE ESTIMATION PARAMETER AND BLOOD PRESSURE MEASUREMENT APPARATUS

(71) Applicant: SEIKO EPSON CORPORATION, Tokyo (JP)

(72) Inventor: Hiromitsu MIZUKAMI, Shiojiri (JP)

(73) Assignee: SEIKO EPSON CORPORATION, Tokyo (JP)

(21) Appl. No.: 13/853,409

(22) Filed: Mar. 29, 2013

(30) Foreign Application Priority Data
Apr. 18, 2012 (JP) 2012-094521

(51) Int. Cl. A61B 8/08; A61B 8/04

(52) U.S. Cl. CPC A61B 8/5223; A61B 8/04
USPC 600/438

(57) ABSTRACT

As different artery blood pressure combination measurement, peripheral blood pressure combination measurement that combines blood vessel diameter measurement to measure a blood vessel diameter as a blood vessel cross-section index value of a central artery as a first artery with peripheral blood pressure measurement to measure blood pressure of a peripheral artery as a second artery is conducted. Then, a parameter for a blood pressure estimation process (for example, a blood vessel hardness parameter or a correlation parameter) that estimates the central aortic blood pressure from the blood vessel diameter of the central artery is corrected by using measurement results of the peripheral blood pressure combination measurement.

### CROSS-REFERENCE TO RELATED APPLICATIONS

This application claims priority to Japanese Patent Application No. 2012-094521 filed on Apr. 18, 2012. The entire disclosure of Japanese Patent Application No. 2012-094521 is hereby incorporated herein by reference.

### BACKGROUND

1. Technical Field

The present invention relates to a method for correcting a blood pressure estimation parameter and the like.

2. Background Technology

An apparatus which measures blood flow, a blood vessel diameter, and blood pressure using ultrasound and an apparatus which measures the elasticity of a blood vessel have been proposed. These apparatuses have characteristics in that non-invasive measurement is possible without imparting pain or an unpleasant feeling to a person being tested.

For example, a technique is disclosed in Patent Document 1 where changes in a blood vessel diameter or changes in a blood vessel cross-sectional area and changes in blood pressure are considered to have a non-linear relationship, and blood pressure is estimated from a stiffness parameter showing the stiffness of a blood vessel and the blood vessel diameter or the blood vessel cross-sectional area.

Japanese Laid-open Patent Publication No. 2004-41382 (Patent Document 1) is an example of the related art.

### SUMMARY

### Problems to Be Solved by the Invention

Central aortic blood pressure known as the blood pressure at the root of the central artery is considered to be able to serve as an index value of arterial sclerosis or cardiovascular disease. For example, in order to estimate the central aortic blood pressure by applying the technique disclosed in Patent Document 1, the above-described stiffness parameter needs to be corrected by measuring the blood pressure of the central artery such as the main artery or the carotid artery. Normally, however, an invasive measurement method in which a catheter is inserted is necessary to measure the blood pressure of the central artery, which causes a problem that the physical burden to a person being tested becomes significant.

Also, as an apparatus for measuring central aortic blood pressure, a central aortic blood pressure monitor has been put to practical use. In such a central aortic blood pressure monitor, the central aortic blood pressure is estimated from the blood pressure waveform of the radial artery at the wrist part, for example. However, since such a central aortic blood pressure monitor is a stationary type of a large size and expensive, it is unsuitable for a person being tested to carry and measure the central aortic blood pressure over a long period of time or to measure the central aortic blood pressure easily whenever a person being tested wants.

Here, the central aortic blood pressure is taken as an example of characteristic blood pressure. However, there are some kinds of pressure other than the central aortic blood pressure that is difficult to measure depending on the kind of the artery. With respect to this kind of artery, it is not easy to correct a necessary parameter for blood pressure estimation, and there are some cases where blood pressure estimation is impossible.

The invention has been made to address the above-described circumstances, and an advantage of the invention is to provide a novel technique for estimating blood pressure.

### Means Used to Solve the Above-Mentioned Problems

According to a first aspect of the invention to achieve the advantage, a method for correcting a blood pressure estimation parameter includes conducting different artery blood pressure combination measurement that combines blood vessel cross-section index value measurement to measure a blood vessel diameter or a blood vessel cross-sectional area (hereinafter, “blood vessel diameter” or “blood vessel cross-sectional area” are collectively referred to as “blood vessel cross-section index value”) of a first artery with blood pressure measurement of a second artery, and correcting a parameter for a blood pressure estimation process that estimates blood pressure of the first artery from a blood vessel cross-section index value of the first artery by using measurement results of the different artery blood pressure combination measurement.

According to another aspect of the invention, a blood pressure measurement apparatus has a blood vessel cross-section index value measurement section that measures a blood vessel cross-section index value of a first artery, a blood pressure measurement section that measures blood pressure of the first artery by conducting a blood pressure estimation process to estimate the blood pressure of the first artery from the blood vessel cross-section index value measured by the blood vessel cross-section index value measurement section, an input section that inputs blood pressure of a second artery, and a correction section that corrects a parameter for the blood pressure estimation process by using the blood vessel cross-section index value measured by the blood vessel cross-section index value measurement section and the blood pressure input by the input section.

Among blood pressure, diastolic blood pressure has characteristics in that its value does not substantially change irrespective of the part through which the artery flows. Therefore, according to the first aspect and the like, the different artery blood pressure combination measurement that combines blood vessel cross-section index value measurement of a first artery with blood pressure measurement of a second artery is conducted. Then, the parameter for the blood pressure estimation process that estimates the blood pressure of the first artery from the blood vessel cross-section index value of the first artery is corrected by using measurement results of the different artery blood pressure combination measurement. Consequently, the parameter for the blood pressure estimation process can be corrected appropriately, and further the blood pressure can be estimated correctly.

According to a second aspect of the invention, in the method for correcting a blood pressure estimation parameter according to the first aspect, the parameter includes a correlation parameter regarding a relationship between a diastolic blood vessel cross-section index value and diastolic blood pressure of the first artery, and the correcting step includes correcting the correlation parameter by using measurement results of the different artery blood pressure combination measurement.

With the second aspect, the parameter for the blood pressure estimation process can be made proper by correcting the correlation parameter regarding the relationship between the diastolic blood vessel cross-section index value and the diastolic blood pressure of the first artery using measurement results of the different artery blood pressure combination measurement.

According to a third aspect of the invention, in the method for correcting a blood pressure estimation parameter according to the first aspect or the second aspect, the parameter includes a blood vessel hardness parameter showing the blood vessel hardness of the first artery. The method further includes conducting same artery blood pressure combination measurement that combines the blood vessel cross-section index value measurement with blood pressure measurement of the first artery. The correcting step includes correcting the blood vessel hardness parameter by using measurement results of the same artery blood pressure combination measurement.

With the third aspect, the same artery blood pressure combination measurement that combines blood vessel cross-section index value measurement with blood pressure measurement of the first artery is conducted. Then, the blood vessel hardness parameter is corrected by using measurement results of the same artery blood pressure combination measurement. Consequently, a value of the blood vessel hardness parameter that reflects the blood vessel hardness of the first artery can be obtained.

According to a fourth aspect of the invention, in the method for correcting a blood pressure estimation parameter according to the first aspect or the second aspect, the parameter includes a blood vessel hardness parameter showing the blood vessel hardness of the first artery, and the correcting step includes setting a prescribed value as the blood vessel hardness parameter.

With the fourth aspect, the parameter for the blood pressure estimation process can be corrected simply by setting a prescribed value as the blood vessel hardness parameter.

According to a fifth aspect of the invention, in the method for correcting a blood pressure estimation parameter according to any one of the first aspect to the fourth aspect, the first artery is a central artery, the second artery is a peripheral artery, and the blood pressure estimation process is a process to estimate blood pressure at a root of a central artery.

With the fifth aspect, in combination with the above-described aspects, the parameter for the blood pressure estimation process to estimate blood pressure at a root of a central artery can be corrected.

### BRIEF DESCRIPTION OF THE DRAWINGS

Referring now to the attached drawings which form a part of this original disclosure:

FIG. 1 is a diagram that explains a method for correcting a central aortic blood pressure estimation parameter;

FIG. 2 is a graph that explains results of measuring blood pressure at different measurement parts;

FIG. 3 is a block diagram that illustrates an example of a functional configuration of an ultrasound blood pressure monitor;

FIG. 4 is a flow chart that illustrates the flow of a main process;

FIG. 5 is a flow chart that illustrates the flow of a process for correcting a blood vessel hardness parameter;

FIG. 6 is a flow chart that illustrates the flow of a process for correcting a correlation parameter; and

FIG. 7 is a flow chart that illustrates the flow of a second main process.

### DETAILED DESCRIPTION OF EXEMPLARY EMBODIMENTS

Hereinafter, an example of preferred embodiments of the invention will be explained with reference to the attached drawings. In the present embodiment, a first artery is a central artery, a second artery is a peripheral artery, and a parameter for a blood pressure estimation process to estimate blood pressure at a root of a central artery is corrected. In the present embodiment, a blood vessel diameter is used as a blood vessel cross-section index value. However, a blood vessel cross-sectional area can be used instead of a blood vessel diameter (in such a case, the “blood vessel diameter” in the following description can be replaced with the “blood vessel cross-sectional area”). It is apparent that embodiments to which the invention can be applied are not limited to the embodiments described below.

### 1. Principles

In the present embodiment, a parameter for a blood pressure estimation process to estimate central aortic blood pressure (hereinafter, referred to as a “central aortic blood pressure estimation parameter”) is corrected. The central aortic blood pressure mainly refers to the blood pressure at the root of the main artery that is a type of the central artery. There are cases where the blood pressure of the carotid artery is considered as the central aortic blood pressure.

The central aortic blood pressure estimation parameter includes a correlation parameter regarding a relationship between a diastolic blood vessel diameter and diastolic central aortic blood pressure, and a blood vessel hardness parameter showing the blood vessel hardness of the central artery. In the present embodiment, these two kinds of parameters are corrected.

Since the central aortic blood pressure is estimated from the blood vessel diameter of the central artery in the blood pressure estimation process, it is necessary to determine the correlation characteristics between the blood vessel diameter of the central artery and the blood pressure of the central artery. The correlation characteristics can be expressed, for example, by a correlation formula that connects the blood vessel diameter of the central artery and the blood pressure of the central artery with a non-linear relationship.

More specifically, the correlation characteristics can be expressed, for example, by the following formula (1) using pressure exerted on the blood vessel of the central artery and a blood vessel diameter at the time of each blood pressure.

P=Pd·exp[β(D/Dd−1)] (1)

Here, β=In(Ps/Pd)/(Ds/Dd−1)

In formula (1), “Ps” is systolic blood pressure, and “Pd” is diastolic blood pressure. Also, “Ds” is a systolic blood vessel diameter which is a blood vessel diameter at the time of systolic blood pressure, and “Dd” is a diastolic blood vessel diameter which is a blood vessel diameter at the time of diastolic blood pressure. Further, “β” is an index value showing a blood vessel hardness called a stiffness parameter.

The blood vessel hardness parameter corresponds to the stiffness parameter “β” in formula (1). The correlation parameter corresponds to the diastolic blood pressure “Pd” and the diastolic blood vessel diameter “Dd”.

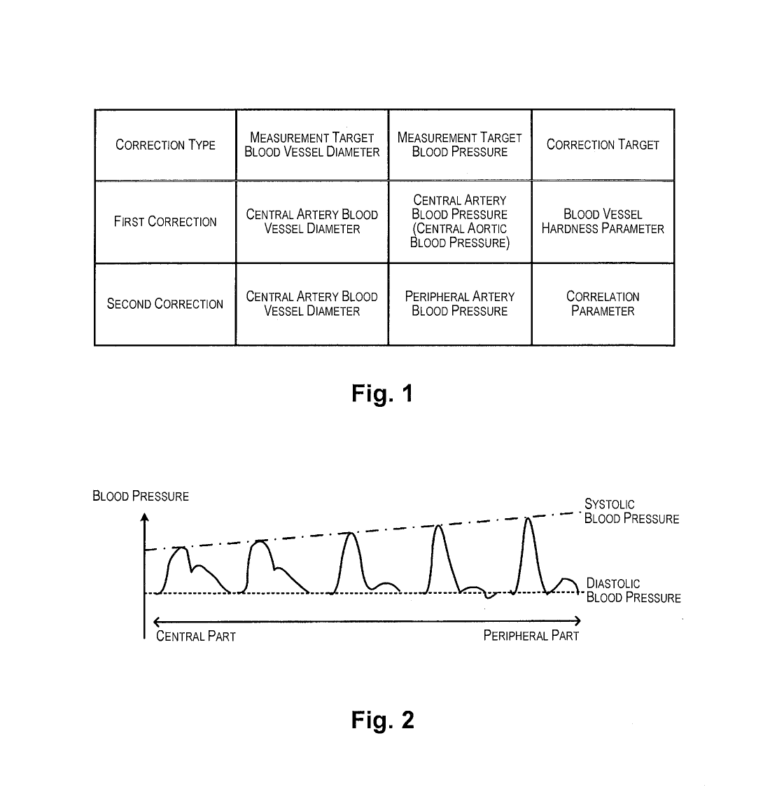

One of the main features of the present embodiment is in that correction of the central aortic blood pressure estimation parameter is divided into correction of the blood vessel hardness parameter and correction of the correlation parameter. More specifically, the correction has two kinds of correction including first correction and second correction as illustrated in FIG. 1.

In the first correction, the blood vessel hardness parameter is corrected by combining measurement of a blood vessel diameter of the central artery with measurement of blood pressure of the central artery (central aortic blood pressure measurement). Hereinafter, this combination is referred to as “central aortic blood pressure combination measurement”. The central aortic blood pressure combination measurement is a type of the same artery blood pressure combination measurement.

In the second correction, the correlation parameter is corrected by combining measurement of a blood vessel diameter of the central artery with measurement of blood pressure of the peripheral artery (peripheral blood pressure measurement). Hereinafter, this combination is referred to as “peripheral blood pressure combination measurement”. The peripheral blood pressure combination measurement is a type of the different artery blood pressure combination measurement.

As described above, in the present embodiment, correction of the blood vessel hardness parameter is conducted by the first correction using the central aortic blood pressure combination measurement, and correction of the correlation parameter is conducted by the second correction using the peripheral blood pressure combination measurement.

The reason to use two kinds of correction will be explained with reference to FIG. 2. FIG. 2 is a graph that shows an example of physiological experimental results of measuring blood pressure at different measurement parts from the central part to the peripheral part of a living body. In FIG. 2, the horizontal axis shows the measurement parts. The measurement part gets closer to the central part as it goes to the left in FIG. 2, and the measurement part gets closer to the peripheral part as it goes to the right in FIG. 2. The vertical axis shows blood pressure. FIG. 2 shows an example of changes in the blood pressure with respect to five measurement parts.

This drawing shows that the systolic blood pressure has a tendency to gradually increase as the measurement part gets close to the peripheral part from the central part. It is considered that this is caused by a so-called peaking phenomenon. The diastolic blood pressure does not change in the central part or in the peripheral part, and has a uniform value.

From these results, there is a high possibility that the blood vessel hardness of the central artery cannot be accurately reflected when the blood vessel hardness parameter is corrected by using the blood pressure of the peripheral artery because the systolic blood pressure is different in the central artery and the peripheral artery. Therefore, correction of the blood vessel hardness parameter is the first correction using the blood pressure of the central artery. Incidentally, for example, a central aortic blood pressure monitor used in medical institutions or the like can be used for measurement of the blood pressure (central aortic blood pressure) of the central artery.

On the other hand, since the blood vessel diameter of the central artery is not uniform and has a slight difference, the correlation characteristics need to be adjusted corresponding to measurement conditions or the like (for example, wearing conditions of the measurement apparatus) at the time of measuring the blood vessel diameter of the central artery. In order to measure the blood pressure of the central artery, a medial institution or the like can devote time and care to visit a person being tested. However, in order to measure the blood pressure of the peripheral artery, blood pressure measurement can be easily conducted at home by using a commercially available blood pressure monitor. Since the diastolic blood pressure does not change in the central artery or in the peripheral artery, correction of the correlation parameter can be conducted sufficiently by measuring the blood pressure of the peripheral artery. Therefore, correction of the correlation parameter is the second correction using the blood pressure of the peripheral artery.

### 2. Embodiment

Next, explanations will be made on an embodiment of a central aortic blood pressure measurement apparatus that estimates central aortic blood pressure by conducting correction of a central aortic blood pressure estimation parameter in accordance with the above-described principles with the radial artery of a person being tested as the peripheral artery and the carotid artery as the central artery. The central aortic blood pressure measurement apparatus is a type of the blood pressure measurement apparatus according to the invention. In the present embodiment, the central aortic blood pressure measurement apparatus is an ultrasound blood pressure monitor 1.

### 2-1. Functional Configuration

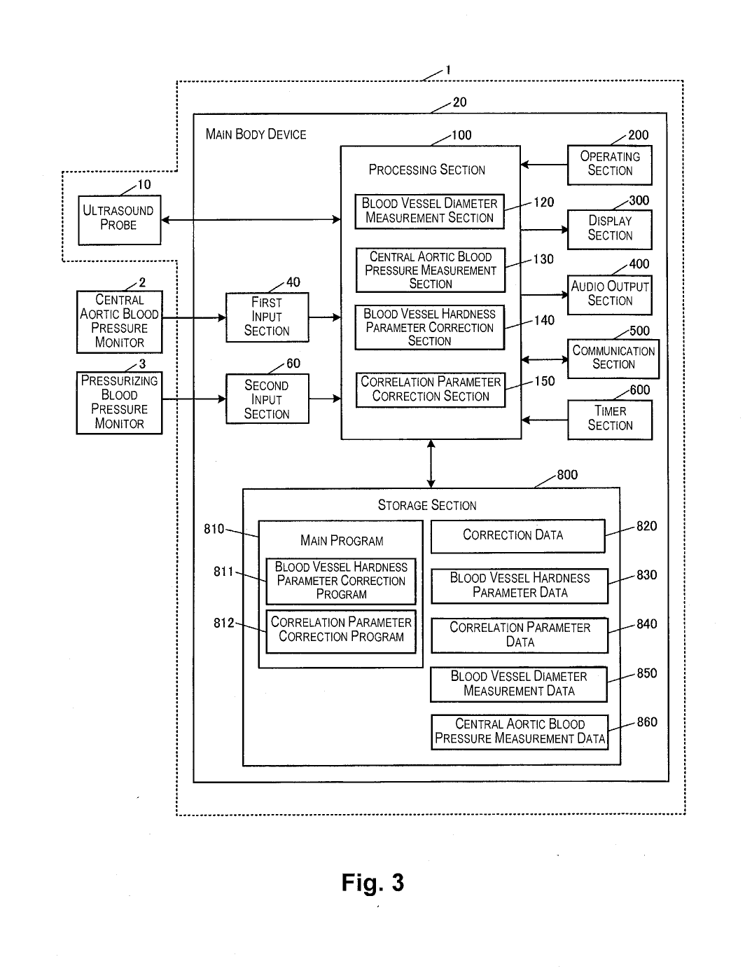

FIG. 3 is a block diagram that illustrates an example of a functional configuration of the ultrasound blood pressure monitor 1. The ultrasound blood pressure monitor 1 has an ultrasound probe 10 and a main body device 20. The ultrasound blood pressure monitor 1 is configured such that measurement results of a central aortic blood pressure monitor 2 and a pressurizing blood pressure monitor 3 can be input by connecting the ultrasound blood pressure monitor 1 to the central aortic blood pressure monitor 2 and the pressurizing blood pressure monitor 3 with a cable.

The ultrasound probe 10 is a small-sized contact that transmits and receives ultrasound by switching an ultrasound transmission mode and an ultrasound reception mode with a time division method in accordance with a control signal output from a blood vessel diameter measurement section 120. A signal received by the ultrasound probe 10 is output to the blood vessel diameter measurement section 120. In the present embodiment, the ultrasound probe 10 is placed at the neck part of a person being tested, and is used to measure the blood vessel diameter of the carotid artery that is the central artery.

The main body device 20 has a first input section 40, a second input section 60, a processing section 100, an operating section 200, a display section 300, an audio output section 400, a communication section 500, a timer section 600, and a storage section 800. The main body device 20 is configured such that a circuit or the like to implement each functional section is housed in a portable small case. It can be said that the main body device 20 is a kind of computer control device.

The first input section 40 connects to the central aortic blood pressure monitor 2 and inputs a measurement value of blood pressure. The central aortic blood pressure monitor 2 is a central aortic blood pressure measurement apparatus that estimates central aortic blood pressure based on the pulse wave of the radial artery measured, for example, at the wrist part of a person being tested.

The second input section 60 connects to the pressurizing blood pressure monitor 3 and inputs a measurement value of blood pressure. The pressurizing blood pressure monitor 3 is a pressurizing blood pressure measurement apparatus that measures blood pressure, for example, by wrapping a cuff band around the upper arm part or the wrist part of a person being tested.

The processing section 100 is a control apparatus and a computation apparatus that comprehensively control each section of the ultrasound blood pressure monitor 1 and is configured to have a microprocessor such as a CPU (Central Processing Unit) or a DSP (Digital Signal Processer), an ASIC (Application Specific Integrated Circuit), and the like.

The processing section 100 has the blood vessel diameter measurement section 120, a central aortic blood pressure measurement section 130, a blood vessel hardness parameter correction section 140, and a correlation parameter correction section 150 as the main functional sections. Here, these functional sections are described only as examples, and it is not necessary that all of these functional sections are essential configuration elements. Also, it is apparent that functional sections other than these can be essential configuration elements.

The blood vessel diameter measurement section 120 controls transmission and reception of ultrasound of the ultrasound probe 10, and measures a blood vessel diameter of a target blood vessel by using a reception signal of reflected ultrasound waves output from the ultrasound probe 10. In the present embodiment, the carotid artery is a target blood vessel. The blood vessel diameter measurement section 120 is a type of a blood vessel cross-section index value measurement section that measures a blood vessel cross-section index value of the central artery. The blood vessel diameter measurement section 120 is configured to be able to continuously measure a blood vessel diameter. As a method for continuously measuring a blood vessel diameter, a phase difference tracking method can be applied, for example.

The central aortic blood pressure measurement section 130 measures central aortic blood pressure by conducting a blood pressure estimation process that estimates central aortic blood pressure from the blood vessel diameter measured by the blood vessel diameter measurement section 120. The blood vessel hardness parameter correction section 140 corrects the blood vessel hardness parameter by using a blood vessel diameter measured by the blood vessel diameter measurement section 120 and blood pressure input by the first input section 40. The correlation parameter correction section 150 corrects the correlation parameter by using a blood vessel diameter measured by the blood vessel diameter measurement section 120 and blood pressure input by the second input section 60.

The operating section 200 is an input apparatus that is configured to have a button switch and the like, and a signal of a pressed button is output to the processing section 100. Various kinds of instructions such as instructions to start measuring central aortic blood pressure is input by operation of the operating section 200.

The display section 300 is a display apparatus that is configured to have an LCD (Liquid Crystal Display) and the like, and conducts various kinds of displays based on a display signal input from the processing section 100. Measurement results and the like of central aortic blood pressure by the central aortic blood pressure measurement section 130 are displayed on the display section 300.

The audio output section 400 is an audio output apparatus that conducts various kinds of audio output based on an audio output signal input from the processing section 100.

The communication section 500 is a communication apparatus for transmitting and receiving information used in the apparatus to and from an external information processing apparatus based on the control of the processing section 100. As the communication method of the communication section 500, it is possible to apply various methods such as a method in which a wired connection is established with a cable in accordance with a prescribed communication standard, a method in which a connection is established via an intermediate apparatus also used as a recharger referred to as a cradle, a method in which a wireless communication is established using short-distance wireless communication. When the connection with the central aortic blood pressure monitor 2 or the pressurizing blood pressure monitor 3 is a communication connection, the first input section 40 and the second input section 60 serve as the communication section 500.

The timer section 600 is a timer apparatus that is configured to have a crystal oscillator and the like constructed by a crystal resonator and an oscillator circuit, and measures time. The time measured by the timer section 600 is output to the processing section 100 as needed.

The storage section 800 is configured to have a storage apparatus such as a ROM (Read Only Memory), a flash ROM, or a RAM (Random Access Memory). The storage section 800 stores a system program of the ultrasound blood pressure monitor 1, various kinds of programs for implementing various kinds of functions such as a blood vessel diameter measurement function, a central aortic blood pressure estimation function, or a correction function, data, and the like. The storage section 800 also has a work area that temporarily stores data during processing, processing results, or the like of various kinds of processing.

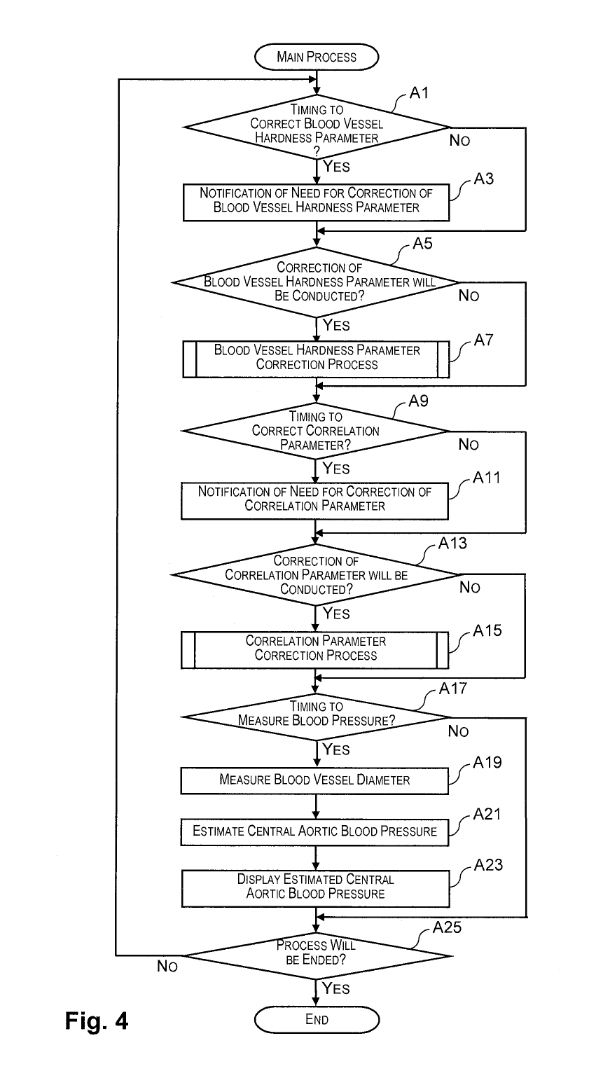

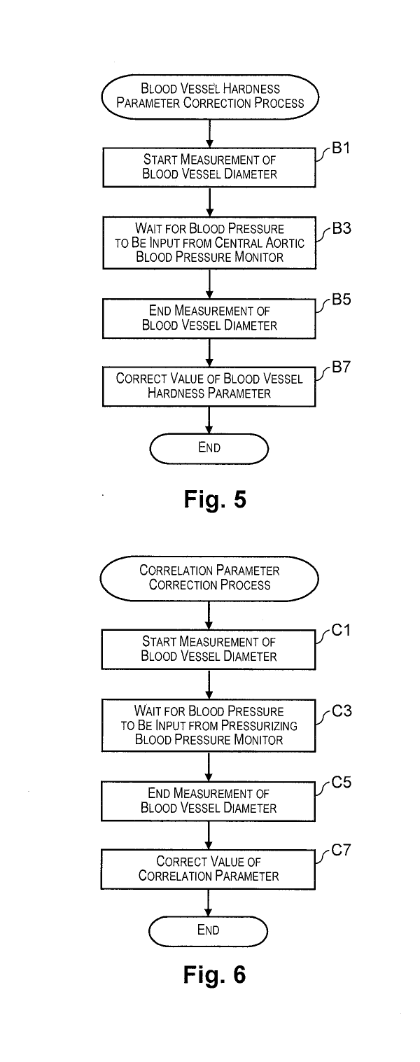

A main program 810 to be read out, for example, by the processing section 100 and executed as a main process (see FIG. 4) is stored in the storage section 800 as a program. The main program 810 includes a blood vessel hardness parameter correction program 811 to be executed as a blood vessel hardness parameter correction process (see FIG. 5) and a correlation parameter correction program 812 to be executed as a correlation parameter correction process (see FIG. 6) as a subroutine. These processes will be described later in detail using a flow chart.

Further, correction data 820, blood vessel hardness parameter data 830, correlation parameter data 840, blood vessel diameter measurement data 850, and central aortic blood pressure measurement data 860 are stored in the storage section 800 as data.

The correction data 820 is data used for correction of the central aortic blood pressure estimation parameter. This includes measurement results of the central aortic blood pressure combination measurement used for correction of the blood vessel hardness parameter and measurement results of the peripheral blood pressure combination measurement used for correction of the correlation parameter.

The blood vessel hardness parameter data 830 is data in which a correction value of the blood vessel hardness parameter is stored, and this data is renewed every time the blood vessel hardness parameter correction process is conducted. The correlation parameter data 840 is data in which a correction value of the correlation parameter is stored, and this data is renewed every time the correlation parameter correction process is conducted.

The blood vessel diameter measurement data 850 is data in which the blood vessel diameter measured by the blood vessel diameter measurement section 120 is stored. The central aortic blood pressure measurement data 860 is data in which the central aortic blood pressure estimated by the central aortic blood pressure measurement section 130.

### 2-2. Process Flow

FIG. 4 is a flow chart that illustrates the flow of the main process executed in accordance with the main program 810 stored in the storage section 800.

First, the processing section 100 determines whether it is a timing to correct the blood vessel hardness parameter (step A1). Various timings can be set as the timing to correct the blood vessel hardness parameter. Although it is considered that the blood vessel hardness parameter changes in a case where an organic change in the blood vessel occurs, it takes a relatively long period of time (several months-years). Thus, a timing when a prescribed period of time (for example, three months) passes since a previous blood vessel hardness parameter correction process is conducted can be set as the correction timing.

When the processing section 100 determines that it is the timing to correct the blood vessel hardness parameter (step A1; Yes), the processing section 100 conducts prescribed notification control to notify a person being tested of the need for correction of the blood vessel hardness parameter (step A3). For example, control is conducted such that a message is displayed on the display section 300 to encourage a person being tested to visit a medical institution or the like and get treatment regarding correction of the blood vessel hardness parameter, or an audio guidance is output from the audio output section 400.

Next, the processing section 100 determines whether correction of the blood vessel hardness parameter will be conducted or not (step A5). For example, the processing section 100 determines whether a person being tested pressed a button to start the correction or not. When the processing section 100 determines that correction of the blood vessel hardness parameter will be conducted (step A5; Yes), the correction of the blood vessel hardness parameter is conducted in accordance with the blood vessel hardness parameter correction program 811 stored in the storage section 800 (step A7).

FIG. 5 is a flow chart that illustrates the flow of a process for correcting the blood vessel hardness parameter. First, the blood vessel diameter measurement section 120 starts measurement of a blood vessel diameter of the carotid artery (measurement of a systolic blood vessel diameter and a diastolic blood vessel diameter) (step B1). Then, the processing section 100 waits for measurement of blood pressure to be input from the central aortic blood pressure monitor 2 (step B3). The blood vessel diameter measurement section 120 continuously measures the blood vessel diameter until the measurement of blood pressure by the central aortic blood pressure monitor 2 ends, and causes the storage section 800 to store as the correction data 820.

When the measurement value of blood pressure is input from the central aortic blood pressure monitor 2 via the first input section 40, the blood vessel diameter measurement section 120 ends the measurement of the blood vessel diameter (step B5). Next, the blood vessel hardness parameter correction section 140 corrects the value of the blood vessel hardness parameter (step B7).

More specifically, a representative value of the blood vessel diameter measured by the blood vessel diameter measurement section 120 is decided until the measurement of blood pressure by the central aortic blood pressure monitor 2 ends. The representative value can be an average value or a median value. The blood vessel hardness parameter (for example, the stiffness parameter “β”) is corrected by using the representative value of the blood vessel diameter (the systolic blood vessel diameter and the diastolic blood vessel diameter) and the measurement value of the blood pressure (the systolic blood pressure and the diastolic blood pressure) by the central aortic blood pressure monitor 2, and causes the storage section 800 to store as the blood vessel hardness parameter data 830. With this, the blood vessel hardness parameter correction process ends.

Returning to the main process of FIG. 4, after the blood vessel hardness parameter correction process is conducted, the processing section 100 determines whether it is a timing to correct the correlation parameter (step A9). As the timing to correct the correlation parameter, a timing when a period of time shorter than that of the timing to correct the blood vessel hardness parameter passes can be set.

In a case where measurement of blood pressure is constantly conducted by the he ultrasound blood pressure monitor 1, it is assumed that the wearing position of the ultrasound probe 10 on the neck will be displaced by body movement of a person being tested. Since the blood vessel diameter of the carotid artery is not uniform and is different depending on the position, there is a possibility that the correlation characteristics between the blood vessel diameter and the blood pressure of the carotid artery will change with a relatively short period. Therefore, correction of the correlation parameter can be conducted at a certain time every day (for example, 8 a.m.), and a timing corresponding to this time can be set as the correction timing.

When the processing section 100 determines that it is the timing to correct the correlation parameter (step A9; Yes), the processing section 100 conducts prescribed notification control to notify a person being tested of the need for correction of the correlation parameter (step A11). For example, prescribed notification control is conducted to encourage a person being tested to conduct correction of the correlation parameter using a household blood pressure monitor own by the person being tested.

Next, the processing section 100 determines whether correction of the correlation parameter will be conducted or not (step A13). For example, the processing section 100 determines whether a person being tested pressed a button to start the correction or not. When the processing section 100 determines that correction of the blood vessel hardness parameter will be conducted (step A13; Yes), the correction of the correlation parameter is conducted in accordance with the correlation parameter correction program 812 stored in the storage section 800 (step A15).

FIG. 6 is a flow chart that illustrates the flow of a process for correcting the correlation parameter. First, the blood vessel diameter measurement section 120 starts measurement of a blood vessel diameter of the carotid artery (step C1). In the measurement of a blood vessel diameter in step C1, it is sufficient that at least a diastolic blood vessel diameter of the carotid artery is measured. Then, the processing section 100 waits for measurement of blood pressure to be input from the pressurizing blood pressure monitor 3 (step C3). The blood vessel diameter measurement section 120 continuously measures the blood vessel diameter until the measurement of blood pressure by the pressurizing blood pressure monitor 3 ends, and causes the storage section 800 to store as the correction data 820.

When the measurement value of blood pressure is input from the pressurizing blood pressure monitor 3 via the second input section 60, the blood vessel diameter measurement section 120 ends the measurement of the blood vessel diameter (step C5). Next, the correlation parameter correction section 150 corrects the value of the correlation parameter (step C7).

More specifically, a representative value of the diastolic blood vessel diameter measured by the blood vessel diameter measurement section 120 is decided until the measurement of blood pressure by the pressurizing blood pressure monitor 3 ends. The representative value can be an average value or a median value. The representative value of the diastolic blood vessel diameter and the measurement value of the diastolic blood pressure by the pressurizing blood pressure monitor 3 are used as the correlation parameter, and causes the storage section 800 to store as the correlation parameter data 840. With this, the correlation parameter correction process ends.

Returning to the main process of FIG. 4, after the correlation parameter correction process is conducted, the processing section 100 determines whether it is a timing to measure blood pressure (step A17). As the timing to measure blood pressure, for example, a timing at prescribed time intervals (for example, every hour) can be used, or a timing when a person being tested gives instructions to measure blood pressure can be used.

When the processing section 100 determines that it is the timing to measure blood pressure (step A17; Yes), the blood vessel diameter measurement section 120 measures the blood vessel diameter of the carotid artery, and causes the storage section 800 to store as the blood vessel diameter measurement data 850 (step A19).

Next, the central aortic blood pressure measurement section 130 conducts a blood pressure estimation process that estimates central aortic blood pressure by using the blood vessel diameter measured in step A19 from a correlation formula determined by the correction value of the blood vessel hardness parameter stored in the blood vessel hardness parameter data 830 and the correction value of the correlation parameter stored in the correlation parameter data 840, and the estimated central aortic blood pressure is stored in the central aortic blood pressure measurement data 860 of the storage section 800 (step A21). Then, the processing section 100 causes the display section 300 to display the estimated central aortic blood pressure (step A23).

Subsequently, the processing section 100 determines whether the process will be ended or not (step A25). When the processing section 100 determines that the process will be continued (step A25; No), the processing section 100 returns the process to step A1. When the processing section 100 determines that the process will be ended (step A25; Yes), the processing section 100 ends the main process.

### 3. Effects

Diastolic blood pressure has characteristics in that its value does not substantially change in the central artery and the peripheral artery. While it is difficult to measure blood pressure in the central artery non-invasively, it is easy to measure blood pressure in the peripheral artery non-invasively. Therefore, as the different artery blood pressure combination measurement, the peripheral blood pressure combination measurement that combines blood vessel diameter measurement of the central artery with blood pressure measurement of the peripheral artery is conducted. Then, the parameter for the blood pressure estimation process that estimates the central aortic blood pressure from the blood vessel diameter of the central artery is corrected by using measurement results of the peripheral blood pressure combination measurement. Consequently, the parameter for the blood pressure estimation process can be corrected appropriately, and further the central aortic blood pressure can be estimated correctly.

According to the present embodiment, in the first correction, as the same artery blood pressure combination measurement, the central aortic blood pressure combination measurement that combines blood vessel diameter measurement of the central artery with blood pressure measurement of the central artery is conducted. Then, the blood vessel hardness parameter (for example, the stiffness parameter “β”) is corrected by using measurement results of the central aortic blood pressure combination measurement. Consequently, the parameter for the blood pressure estimation process that estimates the central aortic blood pressure can be corrected appropriately.

According to the present embodiment, in the second correction, the correlation parameter regarding a relationship between the diastolic blood vessel diameter and the diastolic central aortic blood pressure (for example, the diastolic blood pressure “Pd” and the diastolic blood vessel diameter “Dd”) is corrected by using measurement results of the peripheral blood pressure combination measurement.

### 4. Modified Example

It is apparent that embodiments to which the invention can be applied are not limited to the embodiment described above and appropriate changes are possible in a scope which does not depart from the subject matter of the invention. Hereinafter, modified examples will be described.

### 4-1. Artery

In the above-described embodiment, the first artery is a central artery, the second artery is a peripheral artery, and the blood pressure estimation process is a process to estimate blood pressure at a root of a central artery. However, the combination of arteries that can be selected as the first artery and the second artery is not limited to this. Application of the invention has significance with respect to a combination of arteries that have different values of systolic blood pressure.

For example, the subclavian artery branching from the main artery that is a type of the central artery can be used as the first artery, the peripheral artery such as the radial artery can be used as the second artery, and the parameter for the blood pressure estimation process that estimates the blood pressure of the subclavian artery from the blood vessel diameter of the subclavian artery can be corrected. In this case, the same artery blood pressure combination measurement that combines blood vessel diameter measurement of the subclavian artery with blood pressure measurement of the subclavian artery is conducted. The blood vessel diameter measurement of the subclavian artery can be carried out by using ultrasound, for example. The blood pressure measurement of the subclavian artery can be carried out by using a catheter, for example. Then, the blood vessel hardness parameter (for example, the stiffness parameter) showing the blood vessel hardness of the subclavian artery is corrected by using measurement results of the same artery blood pressure combination measurement.

Also, in order to measure the blood vessel diameter of the subclavian artery, the different artery blood pressure combination measurement that combines with blood pressure measurement of the peripheral artery is conducted. The blood pressure measurement of the peripheral artery can be carried out by measuring blood pressure at the upper arm part or the wrist part with a pressurizing blood pressure monitor, for example. Then, the correlation parameter regarding a relationship between the diastolic blood vessel diameter of the subclavian artery and the diastolic blood pressure of the subclavian artery is corrected by using measurement results of the different artery blood pressure combination measurement.

### 4-2. Blood Vessel Cross-section Index Value

In the above-described embodiment, a blood vessel diameter is used as the blood vessel cross-section index value. However, a blood vessel cross-sectional area can be used as the blood vessel cross-section index value. The correlation characteristics between the blood vessel diameter and the blood pressure can be defined similarly by replacing the blood vessel diameter “D” with a blood vessel cross-sectional area “S”. The blood vessel cross-sectional area can be obtained from a B-mode image by tracing or can be obtained from a blood flow display of a color Doppler method.

### 4-3. Method for Measuring Blood Vessel Diameter

In the above-described embodiment, the method for measuring a blood vessel diameter is a measurement method using ultrasound. However, it is apparent that the method for measuring a blood vessel diameter is not limited to this. For example, it is possible to employ a method for measuring a blood vessel diameter of a target artery by receiving reflected light when light of a prescribed wavelength is emitted from a light emitting element toward a target artery and conducting signal processing.

### 4-4. Central Aortic Blood Pressure Measurement Apparatus

In the above-described embodiment, a central aortic blood pressure measurement apparatus aimed at personal measurement of central aortic blood pressure by a person being tested who can take free action is explained. However, the central aortic blood pressure measurement apparatus to which the invention can be applied is not limited to this. For example, the invention can be applied to an apparatus in which an operator conducts ultrasound diagnosis using an ultrasound probe to a person being tested who is lying down as a medical central aortic blood pressure measurement apparatus.

### 4-5. External Blood Pressure Measurement Apparatus

In the above-described embodiment, a case in which blood pressure of the central artery (central aortic blood pressure) using a central aortic blood pressure monitor that estimates central aortic blood pressure from the pulse wave of the radial artery is explained as an example. However, an apparatus that estimates central aortic blood pressure using another method can be used. Also, for example, an invasive method in which a catheter is inserted into a neck part can be used to measure blood pressure of the central artery instead of a non-invasive method.

In the above-described embodiment, the cuff type pressurizing blood pressure monitor is explained as an example. However, the blood pressure measurement apparatus that measures blood pressure of the peripheral artery is not limited to this. For example, a blood pressure measurement apparatus that measures blood pressure using a tonometry method or a volume-compensation method as a type of a continuous method can be used. A blood pressure measurement apparatus that measures blood pressure using an auscultatory method (a Korotkoff method) as a type of an intermittent method can be used.

### 4-6. Correlation Characteristics

In the above-described embodiment, a case of applying the correlation formula expressed by formula (1) as the correlation formula that shows the correlation characteristics between a blood vessel diameter and blood pressure is described as an example. However, it is apparent that the correlation formula of formula (1) is described only as an example, and another correlation formula can be applied. The kind of the correlation formula can be linear or non-linear.

For example, a correlation formula expressed by formula (2) can be used as a correlation formula in which a blood vessel diameter and blood pressure are approximated using a linear relationship.

P=E×D+B (2)

Here, E=(Ps−Pd)/(Ds−Dd)

B=Pd−E×Dd

In formula (2), “Ps” is systolic blood pressure, and “Pd” is diastolic blood pressure. Also, “Ds” is a systolic blood vessel diameter, and “Dd” is a diastolic blood vessel diameter. Also, “E” is an index value that shows the blood vessel hardness, and “B” is the intercept of the correlation formula.

In a case of applying the correlation formula expressed by formula (2), correction of the central aortic blood pressure estimation parameter can be conducted similarly to the above-described embodiment using the index value “E” that shows the blood vessel hardness as the blood vessel hardness parameter and the intercept “B” of the correlation formula as the correlation parameter.

Incidentally, the storage section 800 does not always need to store data of the correlation formula. It is possible to store data in which the correlation characteristics between a blood vessel cross-section index value (a blood vessel diameter or a blood vessel cross-sectional area) and blood pressure are set in a table format (a lookup table).

### 4-7. Communication Method

In the above-described embodiment, the connection between the ultrasound blood pressure monitor 1 and the external blood pressure measurement apparatus (the central aortic blood pressure monitor 2 and the pressurizing blood pressure monitor 3) is wired. However, another configuration is possible in which a wireless communication section is provided in the ultrasound blood pressure monitor 1 and the external blood pressure measurement apparatus, respectively, and a measurement value of blood pressure is acquired from the external blood pressure measurement apparatus using wireless communication.

### 4-8. Correction Timing

The correction timing of the blood vessel hardness parameter and the correction timing of the correlation parameter described in the above embodiment are examples, and can be modified as appropriate. For example, there are cases where the shape of a measurement target blood vessel of a person being tested changes due to a rapid change in air temperature. Therefore, air temperature during the measurement of blood pressure can be stored and the correction process can be conducted with a timing when a temperature difference between air temperature during the previous measurement and air temperature during the current measurement exceeds a prescribed threshold as the correction timing.

### 4-9. Correction Method

### (1) Correction Procedure

In the above-described embodiment, correction of the blood vessel hardness parameter is conducted as the first correction, and correction of the correlation parameter is conducted as the second correction. However, correction can be conducted as follows.

In the first correction, the blood vessel hardness parameter is corrected and the correlation parameter is initialized using a diastolic blood vessel diameter of the central artery and diastolic blood pressure of the central artery. A correlation formula is set using the correction value of the blood vessel hardness parameter and the initialization value of the correlation parameter.

In the second correction, the correlation parameter is corrected using measurement results of the diastolic blood vessel diameter of the central artery and measurement results of diastolic blood pressure of the peripheral artery. Then, the correlation formula is determined and corrected by shifting the correlation formula set in the first correction so as to pass through a point on a coordinate composed of the correction value of the correlation parameter.

### (2) Setting of Blood Vessel Hardness Parameter

The central aortic blood pressure combination measurement can be omitted, and a prescribed value can be set as the blood vessel hardness parameter. More specifically, for example, a database of average values of the blood vessel hardness parameter are made in advance based on age, gender, physical data, and the like, of a person being tested. Then, correction of the blood vessel hardness parameter can be conducted by allowing the person being tested to input the above-described data, and reading out and setting the value of the blood vessel hardness parameter that corresponds to the input data from the database.

### (3) Correction Process of Blood Vessel Hardness Parameter

The correction process of the blood vessel hardness parameter can be divided into a process of conducting correction of the blood vessel hardness parameter elaborately and a process of conducting correction of the blood vessel hardness parameter simply, and correction of the blood vessel hardness parameter can be conducted by switching these processes.

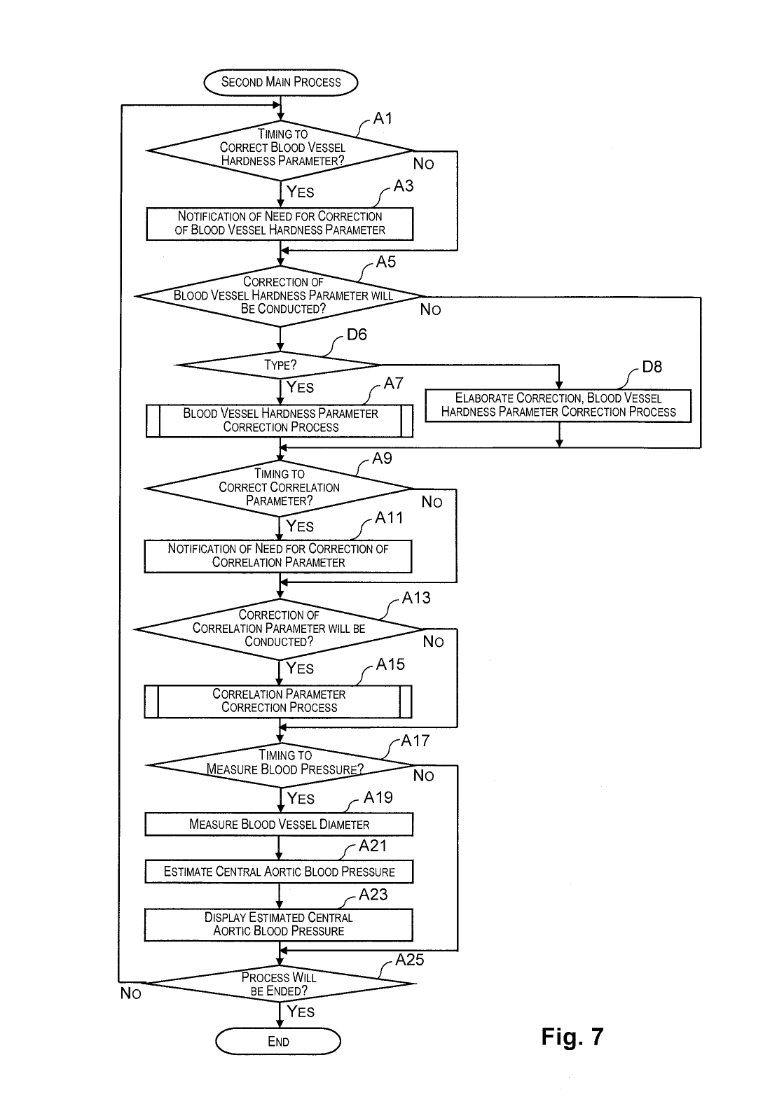

FIG. 7 is a flow chart that illustrates the flow of a second main process executed by the processing section 100 of the ultrasound blood pressure monitor 1 instead of the main process of FIG. 4. The same steps as the main process are given the same reference numerals, and the overlapping explanations are omitted. Then, the explanation focuses on different steps from the main process.

When the processing section 100 determines that correction of the blood vessel hardness parameter will be conducted (step A5; Yes), the processing section 100 determines the type of correction to be conducted (step D6). As the type of correction, two types can be set including “elaborate correction” that corrects the blood vessel hardness parameter by conducting the central aortic blood pressure combination measurement and “simple correction” that corrects the blood vessel hardness parameter without conducting the central aortic blood pressure combination measurement.

When a person being tested selects elaborate correction (step D6; elaborate correction), the processing section 100 conducts the blood vessel hardness parameter correction process explained with reference to FIG. 5 (step A7). In this correction process, the blood vessel hardness parameter is corrected by conducting the central aortic blood pressure combination measurement. Therefore, it can be said that elaborate correction can be achieved.

In contrast, when a person being tested selects simple correction (step D6; simple correction), the processing section 100 conducts a simple blood vessel hardness parameter correction process (step D8). In this correction process, as explained in “(2) Setting of Blood Vessel Hardness Parameter”, for example, the blood vessel hardness parameter is corrected by using values of the blood vessel hardness parameter that have been made a database in advance. In this correction process, the blood vessel hardness parameter is corrected by setting a prescribed value without conducting the central aortic blood pressure combination measurement. Therefore, it can be said that simple correction can be achieved.

The entire disclosure of Japanese Patent Application No. 2012-094521, filed on Apr. 18, 2012, is expressly incorporated by reference herein.

What is claimed is:

1. A method for correcting a blood pressure estimation parameter comprising:
conducting different artery blood pressure combination measurement that combines blood vessel cross-section index value measurement to measure a blood vessel diameter or a blood vessel cross-sectional area (hereinafter, “blood vessel diameter” or “blood vessel cross-sectional area” are collectively referred to as “blood vessel cross-section index value”) of a first artery with blood pressure measurement of a second artery; and
correcting a parameter for a blood pressure estimation process that estimates blood pressure of the first artery from a blood vessel cross-section index value of the first artery by using measurement results of the different artery blood pressure combination measurement.

2. The method for correcting a blood pressure estimation parameter according to claim 1, wherein the parameter includes a correlation parameter regarding a relationship between a diastolic blood vessel cross-section index value and diastolic blood pressure of the first artery, and the correcting step includes correcting the correlation parameter by using measurement results of the different artery blood pressure combination measurement.

3. The method for correcting a blood pressure estimation parameter according to claim 1, wherein the parameter includes a blood vessel hardness parameter showing the blood vessel hardness of the first artery, the method further comprises conducting same artery blood pressure combination measurement that combines the blood vessel cross-section index value measurement with blood pressure measurement of the first artery, and the correcting step includes correcting the blood vessel hardness parameter by using measurement results of the same artery blood pressure combination measurement.

4. The method for correcting a blood pressure estimation parameter according to claim 1, wherein the parameter includes a blood vessel hardness parameter showing the blood vessel hardness of the first artery, and the correcting step includes setting a prescribed value as the blood vessel hardness parameter.

5. The method for correcting a blood pressure estimation parameter according to claim 1, wherein the first artery is a central artery, the second artery is a peripheral artery, and the blood pressure estimation process is a process to estimate blood pressure at a root of a central artery.

6. A blood pressure measurement apparatus comprising:
a blood vessel cross-section index value measurement section that measures a blood vessel cross-section index value of a first artery;
a blood pressure measurement section that measures blood pressure of the first artery by conducting a blood pressure estimation process to estimate the blood pressure of the first artery from the blood vessel cross-section index value measured by the blood vessel cross-section index value measurement section;
an input section that inputs blood pressure of a second artery; and
a correction section that corrects a parameter for the blood pressure estimation process by using the blood vessel cross-section index value measured by the blood vessel cross-section index value measurement section and the blood pressure input by the input section.
